(19) United States
(12) Patent Application Publication
(10) Pub. No.: US 2003/0220654 A1
(43) Pub. Date: Nov. 27, 2003

(54) Method and apparatus for endoscopic delivery

(76) Inventors: Juan Jose Pineda, Columbia, MO (US); Timothy J. Henzel, Columbia, MO (US); Joseph E. Greaves, Columbia, MO (US)

Correspondence Address: Alan H. Norman, Thompson Coburn LLP, One US Bank Plaza, St. Louis, MO, 63101, US

(21) Appl. No.: 10/153,755

(22) Filed: May 21, 2002

(51) Int. Cl. A61B 17/24

(52) U.S. Cl. 606/113

(57) ABSTRACT

A method of delivering a device with an endoscope comprises the steps of: providing an endoscope having a sheath with a hollow passage, a wire assembly slidably positioned within the hollow passage, an actuating member operably connected to a proximal end of the wire assembly, and a flexible loop connected to a distal end of the wire assembly; positioning the flexible loop around at least a portion of the device to be delivered; drawing a retractable portion of the loop into the sheath with a first tensile force; delivering the device to its destination location; and drawing the retractable portion of the loop further into the sheath with a second tensile force. Once the flexible loop has been positioned around at least a portion of the device to be delivered, the retractable portion of the loop is drawn into the sheath with the first tensile force, which is sufficient to tighten the loop around the device so that the device can be effectively delivered to a destination location with the endoscope. However, the first tensile force is insufficient to break the flexible loop. Once the device has been delivered to its destination location, the retractable portion of the loop is drawn further into the sheath with a second tensile force, which is greater than the first tensile force. The second tensile force is sufficient to break the flexible loop, thereby freeing the delivered device from the endoscope at its destination location, e.g., within a patient's small intestine (duodenum).

### BACKGROUND OF THE INVENTION

1. Field of the Invention

This invention relates generally to endoscopes and the use of endoscopes. More particularly, this invention relates to endoscopes incorporating loop type instruments or “snares” and methods of using such endoscopes to move or deliver devices within a patient's body.

2. Related Art

Loop type instruments are used with endoscopes for “snaring” or grasping articles or devices. Such loop type instruments typically comprise a flexible wire that is expanded into the shape of an open loop under free conditions. The flexibility of the loop allows the loop to collapse when withdrawn into the distal end of a sheath. Typically, the loop is connected to the distal end of a control wire, which is moveable axially within the sheath, so that the endoscope can be operated to move the loop back and forth relative to the distal end of the sheath.

Such instruments are commonly used to remove polyps from a patient's colon (a process known as a “polypectomy”). In this process, a surgeon inserts an endoscope into the patient's colon, and a “snare” or similar loop type instrument is extended from the distal end of the endoscope and maneuvered to encircle a polyp to be removed. The surgeon then tightens the loop around the polyp by withdrawing the loop partially into the sheath of the endoscope. The surgeon then runs an electric current through the loop, and the resultant heating of the loop cuts the polyp from the lining of the colon or intestine and cauterizes the resulting wound. The “snares” or loops used in such instruments must be of a electrically conductive metal so that the aforementioned current can be used to remove the polyp.

### SUMMARY OF THE INVENTION

A general object of the present invention is to provide a loop type instrument or “snare” for an endoscope and a method of using the same to move or deliver devices within a patient's body. A related object of the invention is to provide a loop type instrument or “snare” for an endoscope that is less expensive to produce than the metal wire loops of the prior art, such as those designed for use in performing polypectomies that employ electric current. Another object of the invention is to provide a loop type instrument or “snare” for an endoscope that is configured to quickly and efficiently release the device when desired, e.g., once the device has been delivered to its destination location within the patient's body. Still another object of the invention is to provide a loop type instrument or “snare” that will not kink, pinch or otherwise plastically deform when being tightened around the device to be delivered, whether during delivery or when releasing the device after delivery, and that will not damage or otherwise harm the surrounding tissue during delivery or when releasing the device after delivery.

In general, an endoscopic device of the present invention comprises a sheath, a wire assembly, an actuating member and a flexible loop. The sheath has a hollow passage. The wire assembly is slidably positioned within the hollow passage of the sheath. The wire assembly has a proximal end and a distal end. The actuating member is movable between a first position and a second position. The actuating member is operably connected to a proximal end of the wire assembly in a manner to cause the distal end of the wire assembly to move between an extended position and a retracted position relative to the sheath in response to movement of the actuating member between its first and second positions. The flexible loop is connected to the distal end of the wire assembly. At least a portion of the loop is retractable into the sheath when the distal end of the wire assembly is moved toward its retracted position. The loop is made of a non-conductive material. The loop has a frangible portion that is configured to break when a sufficient tensile force is applied to the loop.

A method of delivering a device with an endoscope comprises the steps of: providing an endoscope substantially as described above; encircling a portion of the device to be delivered with the flexible loop of the endoscope; operating the actuating member of the endoscope in a manner to tighten the flexible loop around the device to be delivered; delivering the device to a destination location using the endoscope; and manipulating the actuating member to break the frangible portion of the loop. The actuating member is operated in a manner to cause the distal end of the wire assembly to move toward its retracted position so that a portion of the loop is retracted into the sheath, whereby the loop is tightened around the device. Once tightened, the device is delivered from a starting location to a destination location using the endoscope. Then, the actuating member is manipulated in a manner to move the distal end of the wire assembly further toward its retracted position so that a tensile force is applied to the loop. The tensile force applied to the loop is sufficient to break the frangible portion of the loop.

In another aspect of the invention, a method of delivering a device with an endoscope comprises the steps of: positioning the flexible loop around at least a portion of the device to be delivered by the endoscope; drawing a retractable portion of the loop into the sheath in a manner to tighten the loop around the device; delivering the device from a starting location to a destination location using the endoscope; and drawing the retractable portion of the loop further into the sheath with sufficient tensile force to break the frangible portion of the loop, thereby freeing the delivered device at its destination location.

In still another aspect of the invention, a method of delivering a device with an endoscope comprises the steps of: providing an endoscope having a sheath with a hollow passage, a wire assembly slidably positioned within the hollow passage, an actuating member operably connected to a proximal end of the wire assembly, and a flexible loop connected to a distal end of the wire assembly; positioning the flexible loop around at least a portion of the device to be delivered; drawing a retractable portion of the loop into the sheath with a first tensile force; delivering the device to its destination location; and drawing the retractable portion of the loop further into the sheath with a second tensile force. Once the flexible loop has been positioned around at least a portion of the device to be delivered, the retractable portion of the loop is drawn into the sheath with the first tensile force, which is sufficient to tighten the loop around the device so that the device can be effectively delivered to a destination location with the endoscope. However, the first tensile force is insufficient to break the flexible loop. Once the device has been delivered to its destination location, the retractable portion of the loop is drawn further into the sheath with a second tensile force, which is greater than the first tensile force. The second tensile force is sufficient to break the flexible loop, thereby freeing the delivered device from the endoscope at its destination location.

Further objects, features and advantages of the present invention, as well as the structure and operation of various embodiments of the present invention, are described in detail below with reference to the accompanying drawings.

### BRIEF DESCRIPTION OF THE DRAWINGS

FIG. 1 is a perspective view of an endoscope used in the practice of the present invention;

FIG. 2 is an enlarged, fragmented view of a flexible elastic loop of the endoscope of FIG. 1;

FIG. 3 is an enlarged, fragmented view of the flexible loop of FIG. 2 encircling a portion of a device to be delivered by the endoscope;

FIG. 4 is an enlarged, fragmented view of the flexible loop of FIGS. 2 and 3 with the flexible loop partially retracted into the sheath, whereby the loop is tightened around the device to be delivered; and

FIG. 5 is an enlarged, fragmented view of the flexible loop of FIGS. 2 through 4, with a frangible portion of the loop shown in a broken condition.

Reference characters used in these drawings correspond to reference characters used throughout the Detailed Description of the Preferred Embodiments, which follows. These drawings, which are incorporated in and form a part of the specification, illustrate the embodiments of the present invention and, together with the description, serve to explain the principles of the invention.

### DETAILED DESCRIPTION OF THE PREFERRED EMBODIMENTS

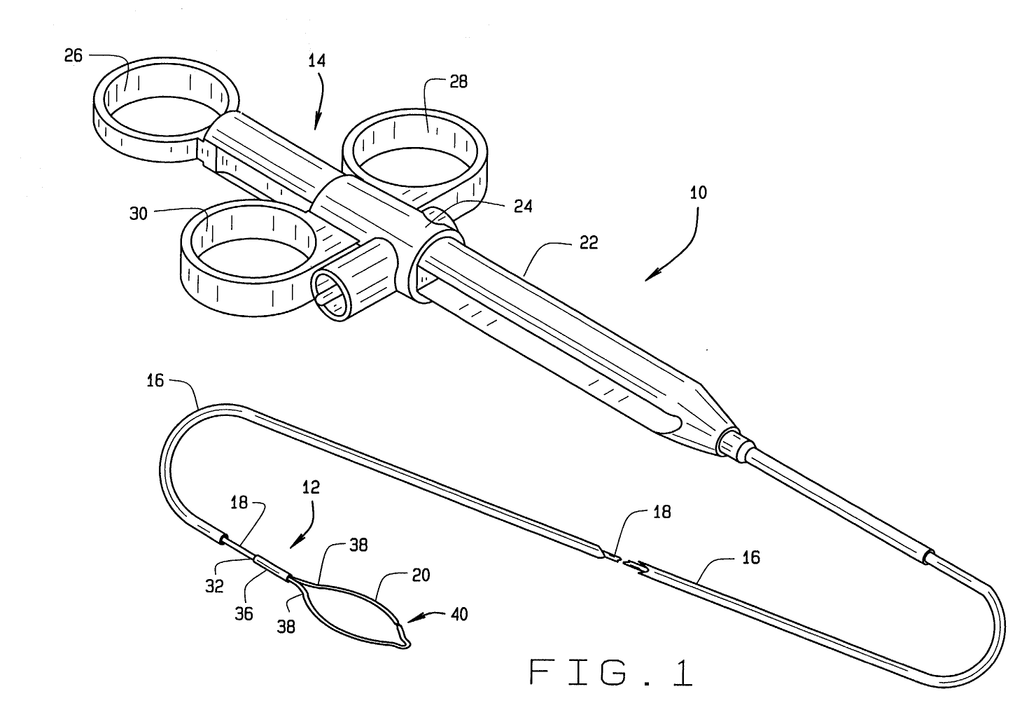

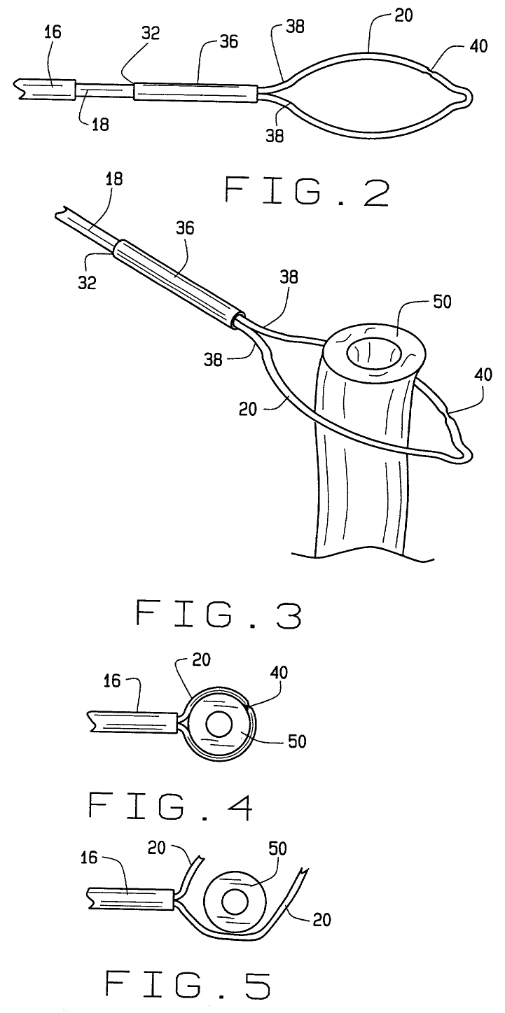

FIG. 1 illustrates an endoscope 10 with a loop type instrument 12 of the present invention. As will be made apparent hereinafter, aside from the loop type instrument 12, the endoscope 10 itself is conventional. In general, the endoscope 10 comprises an actuating assembly 14, a sheath 16, and a control member in the form of a wire assembly 18. The loop type instrument 12 of the present invention comprises a flexible loop 20. The instrument 12 and loop 20 are shown in greater detail in FIGS. 2 through 5, and are described below.

The actuating assembly 14 of the endoscope 10 comprises a stationary portion 22 and a sliding portion 24, which is mounted on the stationary portion 22 in a manner that permits sliding movement of the sliding portion 24 along the length of the stationary portion 22. The stationary portion 22 has a thumb hole 26 and the sliding portion 24 has two finger holes 28 and 30. Thus, a surgeon or other user of the endoscope can place a thumb in the thumb hole 26 and a finger in each of the finger holes 28 and 30 to manually move the sliding portion 24 back and forth along the length of the stationary portion 22. The sliding portion 24 of the actuating assembly 14 is movable between a first position (generally adjacent the thumb hole 26 of the stationary portion) and a second position (generally adjacent the proximal end of the sheath 16).

The endoscope's sheath 16 has a hollow passage, and the wire assembly 18 is slidably positioned within the hollow passage of the sheath 16. The wire assembly 18 has a proximal end operatively connected to the sliding portion 24 of the actuating assembly 14 and a distal end 32 operatively connected to the flexible loop 20. Preferably, the flexible loop 20 is connected to the distal end 32 of the wire assembly 18 with a small ferrule 36. Thus, the distal end 32 of the wire assembly 18 and the flexible loop 20 operatively connected thereto move between a generally extended position and a generally retracted position relative to the sheath 16 in response to movement of the sliding portion 24 of the actuating assembly 14 between its first and second positions. Preferably, at least a proximal portion 38 of the loop 20 is retractable into the distal end of the sheath 16 when the distal end 32 of the wire assembly 18 is moved toward its retracted position, so that the loop can be tightened around an object to be grasped or “snared.”

To this point, the endoscope 10 described is substantially similar to conventional endoscopes of the prior art. However, as described in detail hereinafter, the flexible loop 20 of the present invention is preferably of a non-conductive material and includes a frangible portion 40 that is configured to break when a sufficient tensile force is applied to the loop 20.

FIG. 2 is an enlarged, fragmented view of the flexible loop 20 of the present invention. Again the flexible loop 20 is preferably connected to the distal end 32 of the wire assembly 18 by a small ferrule 36, which is dimensioned to fit within the hollow passage of the sheath 16 when the distal end 32 of the wire assembly 18 is moved toward its retracted position. Preferably, the loop 20 is made of a non-conductive material. More preferably, the loop 20 is made of a non-metallic, polymeric material, such as acetal or another copolymer. Acetal is a high performance engineering polymer, which is also known as polyacetal, polyoxymethylene (POM), or polyformaldehyde. Because of its relatively high strength, modulus, resistance to fatigue, and light weight, acetal is a useful material for the flexible loop 20 of the present invention. Ticona GmbH, one of several manufacturers of acetal copolymer material, sells acetal products under the trademark Celcon®. Alternatively, the flexible loop could be made of another non-conductive, polymeric material, such as nylon or polyester without departing from the scope of the present invention.

The loop 20 is configured to expand into the shape of an open loop under free conditions, i.e., when it is extended from the distal end of the sheath 16. In use, the loop 20 is configured to encircle a device having a diameter larger than a diameter of the hollow passage of the sheath. FIG. 3 is an enlarged, fragmented view of the flexible loop 20 encircling a portion of a device 50 to be delivered by the endoscope 10. As illustrated in FIG. 4, the loop 20 is configured to tighten around the device 50 when the proximal portion 38 of the loop 20 is retracted into the sheath 16 as a result of the distal end 32 of the wire assembly 18 being moved toward its retracted position. Once the loop 20 has been tightened around the device 50 as shown in FIG. 4, the endoscope 10 can be used to move the device in a patients body to deliver the device to a destination location.

When the distal end 32 of the wire assembly 18 is moved toward its retracted position, the loop is subjected to tensile forces. Because the encircled device 50 has a diameter larger than a diameter of the hollow passage of the sheath, the device 50 cannot be retracted into the sheath 16. Thus, as the distal end 32 of the wire assembly 18 is moved toward its retracted position, only the ferrule 36 and proximal portion 38 of the loop 20 can be retracted into the sheath 16, and the rest of the loop 20 tightens around the encircled device 50. As the loop 20 is tightened around the device, it is subjected to a tensile force. The tighter the loop 20 gets, the greater the tensile stress on the loop 20.

As best shown in FIGS. 2 through 4, the loop 20 preferably has a frangible or “weakened” portion 40 that is configured to break when a sufficient tensile force is applied to the loop 20 during use. Preferably, the frangible portion 40 of the loop 20 is configured to withstand a first tensile force that is sufficient to tighten the loop around the device and deliver the device to its destination location, but which is insufficient to break the frangible portion 40. Also, preferably, the frangible portion 40 of the loop 20 is configured to break when subjected to a second tensile force, which is greater than the first tensile force. When subjected to a force at least as great as the second tensile force, the frangible portion 40 of the loop 20 breaks, thereby “opening” the loop and releasing the device 50 from its grasp (see FIG. 5). The magnitude of the second tensile force necessary to cause the frangible portion 40 of the loop 20 to break will depend on the particular materials used and the cross-sectional dimensions or gauge of the loop 20 itself, and particularly the cross-sectional dimensions of the frangible portion 40 of the loop. As discussed below, the most preferred tensile strength of the loop 20 and its frangible portion 40 will differ depending on the particular application. However, the selection of an appropriate loop material and size would be within the ordinary skill in the art.

A method of the present invention is contemplated for delivering a device with an endoscope. In general, the method comprises the steps of: providing an endoscope; encircling a portion of the device (such as the device 50 shown in FIGS. 2 through 5) to be delivered with a flexible loop of the endoscope; operating an actuating assembly of the endoscope in manner to cause a portion of the loop to be retracted into a sheath of the endoscope, whereby the loop is tightened around the device; delivering the device from a starting location to a destination location using the endoscope; and manipulating the actuating assembly of the endoscope in a manner to apply a tensile force to the loop, the tensile force being sufficient to break a frangible portion of the loop. Preferably, the endoscope is substantially similar to the endoscope 10 described above and shown in FIG. 1. Preferably, the step of operating an actuating assembly includes operating the actuating assembly 14 of the endoscope 10 in manner to cause the distal end 32 of the wire assembly 18 to move toward a retracted position so that a portion of the loop 20 is retracted into the sheath 16, whereby the loop 20 is tightened around the device 50. Also, preferably, the step of manipulating the actuating assembly includes manipulating the actuating assembly 14 of the endoscope 10 in a manner to move the distal end 32 of the wire assembly 18 further toward its retracted position so that a tensile force is applied to the loop 20, the tensile force being sufficient to break the frangible portion 40 of the loop 20.

Preferably, the step of manipulating the actuating assembly 14 is performed after the steps of encircling the device 50 and operating the actuating assembly 14. Also, preferably, the step of operating the actuating assembly 14 to tighten the loop 20 around the device 50 is performed with the device 50 at its starting location, and wherein the step of manipulating the actuating assembly 14 to break the frangible portion 40 of the loop 20 is performed with the device 50 at its destination location.

In a preferred embodiment of this method, the step of operating the actuating assembly 14 to tighten the loop 20 around the device 50 includes drawing the proximal portion 38 of the loop 20 into the sheath 16 with a first tensile force, which is sufficient to tighten the loop 20 around the device 50 so that the device can be effectively delivered to its destination location with the endoscope 10, but which is insufficient to break the frangible portion 40 of the loop 20. Preferably, the step of manipulating the actuating assembly 14 includes drawing the proximal portion 38 of the loop 20 into the sheath 16 with a second tensile force (greater than the first tensile force), which is sufficient to break the frangible portion 40 of the loop 20, thereby “opening” the loop 20 and releasing the device 50 from its grasp at the destination location. Again, the magnitude of the second tensile force necessary to cause the frangible portion 40 of the loop 20 to break will depend on the particular materials used and the cross-sectional dimensions of the frangible portion 40 of the loop 20, and the most preferred tensile strength of the loop 20 and its frangible portion 40 will depend on the particular application.

The inventors herein have determined that the apparatus and methods of the present invention are particularly useful in endoscopic delivery of a feeding tube into a patient's duodenum. Such feeding tubes are needed to feed patients who cannot swallow for one reason or another. This procedure is generally less invasive, less risky and less costly than a classical surgical gastrostomy for inserting a feeding tube, which requires opening of the abdomen. In applying the method of the present invention for this purpose, an endoscope 10, substantially as described above, is used to delivering one end of a feeding tube (represented somewhat schematically by the reference numeral 50 in FIGS. 3 through 5) to a destination location within the patient's duodenum. First, the surgeon uses the loop 20 of the endoscope 10 to encircle one end of the feeding tube 50 (see FIG. 3). Then, the surgeon operates the actuating assembly 14 of the endoscope 10 in manner to cause the proximal portion 38 of the loop 20 to retract into a sheath 16 of the endoscope 10, whereby the loop 20 is tightened around the tube 50 under the first tensile force, which is sufficient to tighten the loop 20, but insufficient to break the frangible portion 40 of the loop 20 (see FIG. 4). Then, the surgeon delivers the end of the tube to a destination location, e.g., in the small bowel (second or third portion of the duodenum). Next, the surgeon manipulates the actuating assembly 14 of the endoscope 10 in a manner to apply a second tensile force to the loop 20, which is sufficient to break the frangible portion 40 of the loop 20, thereby “opening” the loop 20 and releasing the tube from its grasp at the destination location, e.g., small bowel (second or third portion of the duodenum) (see FIG. 5). Finally, the surgeon withdraws the endoscope 10 from the patient.

The inventors herein have also determined that, in using the apparatus and methods of the present invention for endoscopic delivery of a feeding tube into a patient's duodenum, the second tensile force is preferably between about 0.30 lbs. and 1.00 lbs. of force. A tensile force below the lower end of this range may be problematic because the loop 20 may be inadvertently broken during delivery of the tube, and a tensile force above the higher end of this range may only make it unnecessarily difficult for the surgeon to break the loop 20 when desired, which defeats the purpose of the frangible portion 40. If the apparatus and methods of the present invention are used for delivering other devices within a patient's body, the preferred second tensile force may be less than 0.30 lbs. of force or more than 1.00 lbs. of force without departing from the scope of the present invention, depending on the nature of the device to be delivered and depending on difficulty of the path to be navigated by the device during delivery. In using the apparatus and methods of the present invention for endoscopic delivery of a feeding tube into a patient, where the preferred second tensile force is between about 0.30 lbs. and 1.00 lbs. of force, the inventors have determined that the preferred line thickness for a flexible loop made of acetal or a similar copolymer is between about 0.008″ and 0.015″, though thicknesses falling outside this range could be used without departing from the scope of the invention.

As discussed above, the flexible loop 20 of the present invention is preferably made of a non-conductive, non-metallic, polymeric material, such as acetal. Flexible loops used in prior art endoscopes (such as those used in performing polypectomies) have always been made of conductive metallic materials, which is critical so that an electric current can be passed through the loops to cut polyps from the lining of the intestines and cauterize the resulting wounds. While such metal wire loops have been useful in performing polypectomies and similar procedures that employ electric current, the metal wire loops themselves are not ideal for use in practicing the present invention, which has no need for an electric current or cauterization. Metal wire loops are generally heavier and more expensive to produce than the polymeric loops of the present invention. Metal wire loops are also generally tougher to break (when desired) under tensile force than polymeric loops. Also, metal wire loops and other metal devices used in endoscopic procedures for grasping have a greater tendency to kink, pinch or otherwise plastically deform, or to damage or injure the intestinal mucosa, when being tightened around the device to be delivered, or when releasing the device after delivery, unlike the polymeric loops of the present invention, which generally have a greater elastic limit. Yet another problem with metal wire loops or similar metal devices used for grasping, e.g., retrieval forceps, is that they may have a greater tendency to pinch or otherwise damage the tube or other device during delivery to surrounding tissue organs, e.g., intestinal mucosa. Thus, for these reasons and others, the polymeric loops of the present invention represent a better solution than their metallic counterparts for practicing the methods of the present invention, without introducing toxic or otherwise harmful material into the intestinal tract.

In view of the foregoing, it will be seen that the several advantages of the invention are achieved and attained. The embodiments were chosen and described in order to best explain the principles of the invention and its practical application to thereby enable others skilled in the art to best utilize the invention in various embodiments and with various modifications as are suited to the particular use contemplated. As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. An endoscopic device comprising:
a sheath having a hollow passage;
a control member slidably positioned within the hollow passage of the sheath, the control member having a proximal end and a distal end;
an actuating member moveable between a first position and a second position, the actuating member being operatively connected to the proximal end of the control member in a manner to cause the distal end of the control member to move between an extended position and a retracted position relative to the sheath; and
a flexible loop connected to the distal end of the control member, at least a portion of the loop being retractable into the sheath when the distal end of the control member is moved toward its retracted position, the loop being made of a non-conductive material, the loop having a frangible portion that is configured to break when a sufficient tensile force is applied to the loop.

2. The endoscopic device of claim 1 wherein the loop is made of a non-metallic material.

3. The endoscopic device of claim 2 wherein the loop is made of a polymeric material.

4. The endoscopic device of claim 1 wherein the loop is configured to encircle a device having a diameter larger than a diameter of the hollow passage of the sheath, the loop being configured to tighten around the device when the retractable portion of the loop is retracted into the sheath as a result of the distal end of the control member being moved toward its retracted position under a first tensile force that is sufficient to tighten the loop around the device but insufficient to break the frangible portion of the loop, the frangible region of the loop being configured to break, and thereby release the device, when the distal end of the control member is moved further toward its retracted position under a second tensile force greater than the first tensile force.

5. A method of delivering a device with an endoscope, the method comprising the steps of:
providing an endoscope comprising a sheath having a proximal end, a distal end and a hollow passage extending from the proximal end to the distal end, and a flexible loop adapted to extend out the distal end of the sheath, at least a portion of the loop being retractable into the distal end of the sheath, the loop having a frangible portion configured to break when a sufficient tensile force is applied to the loop;
encircling a portion of the device to be delivered with the flexible loop;
retracting a portion of the loop into the distal end of the sheath to cause the loop to tighten around the device;
delivering the device from a starting location to a destination location using the endoscope while the loop is tightened around the device; and
further retracting the portion of the loop into the distal end of the sheath so that a tensile force is applied to the loop, the tensile force being sufficient to break the frangible portion of the loop.

6. The method of claim 5 wherein the step of further retracting the portion of the loop into the distal end of the sheath so that a tensile force is applied to the loop is performed after the steps of encircling a portion of the device to be delivered with the flexible loop and retracting a portion of the loop into the distal end of the sheath to cause the loop to tighten around the device.

7. The method of claim 5 wherein the step of retracting a portion of the loop into the distal end of the sheath to cause the loop to tighten around the device is performed with the device at its starting location, and wherein the step of further retracting the portion of the loop into the distal end of the sheath so that a tensile force is applied to the loop is performed with the device at its destination location.

8. The method of claim 5 wherein the step of retracting a portion of the loop into the distal end of the sheath to cause the loop to tighten around the device includes drawing a portion of the loop into the distal end of the sheath with a first tensile force, the first tensile force being sufficient to tighten the loop around the device so that the device can be effectively delivered to its destination location with the endoscope, but the first tensile force being insufficient to break the frangible portion of the loop.

9. The method of claim 8 wherein the step of further retracting the portion of the loop into the distal end of the sheath so that a tensile force is applied to the loop includes drawing the portion of the loop into the distal end of the sheath with a second tensile force greater than the first tensile force, the second tensile force being sufficient to break the frangible portion of the loop.

10. The method of claim 5 wherein the step of providing an endoscope includes the step of providing an endoscope with a flexible loop made of a non-conductive material.

11. A method of delivering a device with an endoscope, the endoscope having a sheath with a hollow passage, a wire assembly slidably positioned within the hollow passage of the sheath, an actuating member operatively connected to a proximal end of the wire assembly, and a flexible loop connected to a distal end of the wire assembly, at least a portion of the loop being retractable into the sheath, the loop having a frangible portion, the method comprising the steps of:
positioning the flexible loop around at least a portion of the device to be delivered by the endoscope;
drawing the retractable portion of the loop into the sheath in a manner to tighten the loop around the device;
delivering the device from a starting location to a destination location using the endoscope; and
drawing the retractable portion of the loop further into the sheath with sufficient force to break the frangible portion of the loop, thereby freeing the delivered device at its destination location.

12. The method of claim 11 wherein the step of drawing the retractable portion of the loop into the sheath in a manner to tighten the loop around the device is performed by operating the actuating member in manner to cause the distal end of the wire assembly to move in the direction of the proximal end of the wire assembly.

13. The method of claim 11 where the step of delivering the device from a starting location to a destination location is performed after the steps of positioning the flexible loop around the device and drawing the retractable portion of the loop into the sheath to tighten the loop around the device.

14. The method of claim 11 wherein the step of drawing the retractable portion of the loop into the sheath to tighten the loop around the device is performed with the device at its starting location, and wherein the step of drawing the retractable portion of the loop further into the sheath to break the frangible portion of the loop is performed with the device at its destination location.

15. The method of claim 11 wherein the step of drawing the retractable portion of the loop into the sheath in a manner to tighten the loop around the device includes drawing the retractable portion of the loop into the sheath with a first tensile force, the first tensile force being sufficient to tighten the loop around the device so that the device can be effectively delivered to its destination location with the endoscope, but the first tensile force being insufficient to break the frangible portion of the loop.

16. The method of claim 15 wherein the step of drawing the retractable portion of the loop further into the sheath to break the frangible portion of the loop includes drawing the retractable portion of the loop into the sheath with a second tensile force greater than the first tensile force, the second tensile force being sufficient to break the frangible portion of the loop.

17. A method of delivering a device with an endoscope, the method comprising the steps of:
providing an endoscope comprising:
a sheath with a hollow passage;
a wire assembly slidably positioned within the hollow passage of the sheath;
an actuating member operatively connected to a proximal end of the wire assembly; and
a flexible loop connected to a distal end of the wire assembly, at least a portion of the loop being retractable into the sheath;
positioning the flexible loop around at least a portion of the device to be delivered by the endoscope;
drawing the retractable portion of the loop into the sheath with a first tensile force that is sufficient to tighten the loop around the device so that the device can be effectively delivered to a destination location with the endoscope, the first tensile force being insufficient to break the flexible loop;
delivering the device to its destination location using the endoscope; and
drawing the retractable portion of the loop further into the sheath with a second tensile force that is greater than the first tensile force, the second tensile force being sufficient to break the flexible loop, thereby freeing the delivered device from the endoscope at its destination location.

18. The method of claim 17 wherein the step of providing an endoscope with a flexible loop includes providing an endoscope with a flexible loop having a frangible portion that is capable of withstanding the first tensile force but not the second tensile force.

19. The method of claim 17 wherein the step of providing an endoscope with a flexible loop includes providing an endoscope with a flexible loop made of a non-conductive material.

20. The method of claim 17 wherein the device to be delivered is a feeding tube and wherein the step of delivering the device includes delivering at least a portion of the feeding tube to a location within a patient's stomach.
